(19) United States
(12) Patent Application Publication
(10) Pub. No.: US 2007/0048250 A1
(43) Pub. Date: Mar. 1, 2007

(54) Methods of treating concomitant constipation in a cancer treatment regimen including imexon

(75) Inventors: Evan M. Hersh, Tucson, AZ (US); Kathryn A. Grenier, Tucson, AZ (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: AmpliMed Corporation, Tucson, AZ (US)

(21) Appl. No.: 11/512,998

(22) Filed: Aug. 29, 2006

Related U.S. Application Data
(60) Provisional application No. 60/713,236, filed on Aug. 31, 2005.

(51) Int. Cl. A61K 31/715; A61K 31/7072; A61K 31/337

(52) U.S. Cl. 424/78.3; 514/54; 514/49; 514/449

(57) ABSTRACT

Methods of treating imexon-induced constipation in patients in conjunction with their participation in a cancer treatment regimen are disclosed herein. Embodiments of the invention include the treatment of imexon-induced constipation arising from the administration of imexon alone or in combination with other chemotherapeutic agents. Also disclosed are pharmaceutical compositions and pharmaceutical kits comprising a therapeutically effective amount of imexon and an amount of a laxative effective in treating imexon-induced constipation. Embodiments of the present invention include prophylactic anticonstipation regimens comprising one or more laxatives that may be prescribed and/or administered prior to or during the course of treatment of the patient with imexon.

### CROSS REFERENCE TO RELATED APPLICATIONS

This application claims the benefit of U.S. provisional patent application No. 60/713,236, filed Aug. 31, 2005, the disclosure of which is hereby incorporated by reference in its entirety for all purposes.

### FIELD OF THE INVENTION

This disclosure relates generally to methods of treating constipation, and to pharmaceutical kits including instructions to treat constipation, arising in association with the administration to patients, as part of a cancer treatment regimen, of pharmaceutical compositions comprising imexon.

### BACKGROUND INFORMATION

Cancer is a leading cause of death in humans and animals. As such, considerable resources are invested in the discovery and clinical investigation of compounds and compositions that may be effective in treating the many forms of cancer. Cancer chemotherapeutic agents are generally administered to patients as part of a cancer treatment regimen, wherein one or more chemotherapeutic agents is administered at a defined dose and schedule, and the patient's disease and drug-related toxicities are periodically assessed to gauge the need for modifications to the patient's treatment.

Unfortunately, most, if not all, current cancer drug therapies, while effective in varying degrees at treating the underlying cancer, also produce undesirable toxicities in patients. The severity of the toxicities observed in conjunction with the administration of a cancer treatment regimen may be a function of many factors, including the specific chemotherapeutic agent being administered, the dose and dosing schedule of the cancer treatment regimen, the patient's underlying health, and the stage of the patient's disease. In contrast, the type of toxicity observed (e.g., nausea, alopecia, hypertension, etc.) is principally a function of the characteristics of the type of chemotherapeutic agent being administered, including the compound's chemical structure and its mechanism of action.

While a limited range of toxicities (e.g., myelosuppression) are quite common in conjunction with the use of many types of cancer chemotherapeutic agents, the observed toxicities vary in large part from one type of chemotherapeutic agent to another. As such, one may expect to observe toxicities in patients treated with a particular investigational chemotherapeutic agent that are consistent with those observed in patients treated with known chemotherapeutic agents sharing characteristics with the investigational compound. For example, the class of cancer chemotherapeutic agents known as vinca alkaloids, including for example teniposide, vinblastine, vincristine, and vinorelbine, commonly cause constipation in patients being treated with these compounds as part of a cancer treatment regimen. Similarly, patients undergoing a cancer treatment regimen utilizing the platinum-based antineoplastic agents, including for example cisplatin, carboplatin, and oxaliplatin, commonly exhibit neurotoxicity in conjunction with their treatment. With reference to the foregoing examples, one may expect that the administration to a patient of a chemotherapeutic agent sharing chemical characteristics with the vinca alkaloids or with the platinum-based antineoplastic agents, would exhibit constipation or neurotoxicity, respectively, in conjunction with their treatment.

Imexon (4-imino-1,3-diazabicyclo[3.1.0]-hexan-2-one) has the following chemical structure:

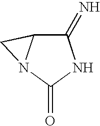

With its aziridine moiety, imexon belongs to a class of cancer chemotherapeutic agents that are known to induce nausea, vomiting, anorexia, and myelosuppression as the major toxicities associated with their administration in a cancer treatment regimen. In addition, many aziridine-containing cancer chemotherapeutic agents, including for example mitomycin C, triethylenemelamine, and diaziquone, among others, also induce gastrointestinal hypermotility/diarrhea as a side effect of their administration.

Imexon has both direct cytotoxicity to tumor cells and immunomodulatory effects. It is active against a variety of human tumor cell lines in culture and agairist fresh human tumor cell lines in clonogenic assay, including selective cytotoxicity to multiple myeloma in the clonogenic assay (S. E. Salmon and E. M. Hersh, J. Natl. Cancer Inst., 86, 228, 1994). In addition, imexon is active against a variety of transplanted tumors in rodents (U. Bicker and G. Hebold, IRCS Med. Sci.: Cancer; Hematology; Immunology and Allergy; Pharmacology, 5, 428, 1977; U. Bicker and P. Fuhse, Exp. Path. Bd. 10, S. 279-284, 1975), and is active against human lymphoma, melanoma, and prostate cancer cell lines in SCID mice (Hersh, et al., Proc. Am. Assoc. Cancer Res., 36, 294, 1995). Objective responses have been observed in dogs with mast cell tumors after treatment with imexon (R. T. Dorr, J. D. Liddil, M. K. Klein, and E. M. Hersh, Invest. New Drugs, 13, 113, 1995), and imexon has demonstrated potential value in combination chemotherapy.

Imexon was developed by Bicker (Immune Modulation and Control of Neoplasia by Adjuvant Therapy, M. A. Ghirigos, Ed., Raven Press, New York, 1978, p. 389) in an investigation of cyanoaziridine derivatives with potential cancerostatic action, and was claimed in U.S. Pat. No. 4,083,987 to Bicker et al. (Apr. 11, 1978). Preparation of imexon and related aziridine-1-carboxamides is described in U.S. Pat. No. 6,476,236 to Remers et al. (Nov. 5, 2002), which is incorporated herein by reference in its entirety. Despite imexon's long history of investigation for potential therapeutic use, previous human clinical studies have given no indication that the observation of a constipation toxicity would be expected in conjunction with the administration of imexon in a cancer treatment regimen. Moreover, pathologic examinations of rats and dogs given imexon in numerous studies have not shown evidence of gastrointestinal lesions.

### SUMMARY OF THE INVENTION

The present invention is directed to the unexpected finding that the administration of imexon in the course of a cancer treatment regimen induces constipation in a large percentage of patients. The occurrence of this toxicity necessitates a treatment, which, in one embodiment, may comprise a prophylactic anticonstipation regimen implemented prior to or during the course of treatment of the patient with imexon.

In one aspect, the present invention provides a method of treating imexon-induced constipation in patients undergoing a cancer treatment regimen in which the cancer treatment regimen includes the administration of a therapeutically effective amount of a pharmaceutical composition comprising imexon. The accompanying constipation may be treated, in one embodiment, via a prophylactic anticonstipation regimen including at least one laxative, the anticonstipation regimen being prescribed or administered in association with the patient's cancer treatment regimen.

In another aspect, the present invention provides a method of treating imexon-induced constipation in a patient being treated with imexon, said method comprising the administration of a laxative sufficient to treat the imexon-induced constipation.

In another aspect, the present invention provides a method of treating constipation in patients undergoing a cancer treatment regimen in which the cancer treatment regimen comprises a combination therapy that includes, as one component of the combination therapy, the administration of a therapeutically effective amount of a pharmaceutical composition comprising imexon. The accompanying constipation may be treated, in one embodiment, via a prophylactic anticonstipation regimen including at least one laxative, the anticonstipation regimen being prescribed or administered in association with the patient's cancer treatment regimen.

In another aspect, the present invention provides a pharmaceutical kit including a therapeutically effective amount of a pharmaceutical composition comprising imexon, instructions for using the pharmaceutical composition in a cancer treatment regimen, and instructions to treat constipation as an event arising in conjunction with the use of the pharmaceutical composition in the cancer treatment regimen. The instructions to treat constipation include, in one embodiment, instructions to prescribe and/or administer at least one laxative.

In another aspect, the present invention provides a pharmaceutical composition including imexon and a laxative.

In another aspect, the present invention provides a pharmaceutical kit including a first pharmaceutical composition and a second pharmaceutical composition. The first pharmaceutical composition includes imexon and the second pharmaceutical composition includes a laxative.

### DETAILED DESCRIPTION OF EMBODIMENTS OF THE INVENTION

Embodiments of methods for treating constipation in patients in conjunction with their participation in a cancer treatment regimen that includes the administration of a therapeutically effective amount of a pharmaceutical composition comprising imexon are disclosed herein. Also disclosed are pharmaceutical kits comprising a therapeutically effective amount of a pharmaceutical composition including imexon, instructions for using the pharmaceutical composition in a cancer treatment regimen, and instructions to treat constipation arising in conjunction with the cancer treatment regimen. In one embodiment, the pharmaceutical kit may include printed material identifying constipation as an adverse reaction that can arise in association with the administration of imexon, and a recommendation to prescribe an anticonstipation regimen to treat the constipation. In the following description, numerous specific details are provided, such as the identification of various components and structures, to provide a thorough understanding of embodiments of the invention. One skilled in the art will recognize however, that the invention can be practiced without one or more of the specific details, or with other methods, components, materials, and the like. In still other instances, well-known components, materials, or processes are not shown or described in detail to avoid obscuring aspects of various embodiments of the invention.

Reference throughout this specification to “one embodiment” or “an embodiment” means that a particular feature, structure, component, or characteristic described in connection with the embodiment is included in at least one embodiment of the present invention. Thus, the appearance of the phrases “in one embodiment” or “in an embodiment” in various places throughout this specification are not necessarily all referring to the same embodiment. Furthermore, the particular features, structures, components, or characteristics may be combined in any suitable manner in one or more embodiments.

I. Definitions

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “therapeutically effective amount” refers to the amount of a compound or composition effective to yield the desired therapeutic response. The therapeutically effective amount may vary with such factors as the particular condition being treated, the physical condition of the patient, the duration of the treatment, the nature of concurrent therapy (if any), and the specific formulations employed.

As used herein, the term “carrier” is a pharmaceutically acceptable solvent, suspending agent or vehicle for delivering imexon or other combination therapy chemotherapeutic agents to the patient. The carrier may be liquid or solid and is selected with regard to the planned manner of administration.

As used herein, “imexon” refers to an unsubstituted 4-imino-1,3-diazabicyclo[3.1.0]-hexan-2-one, or a pharmaceutically acceptable salt, solvate, or prodrug thereof.

As used herein, a “pharmaceutically acceptable salt” refers to, and is intended to include, acid addition salts of the active compound that are prepared with relatively nontoxic acids. Examples of pharmaceutically acceptable acid addition salts include those derived from inorganic and organic acids, including, but not limited to, chlorides, bromides, sulfates, nitrates, phosphates, sulfonates, formats, tartrates, maleates, malates, citrates, benzoates, salicylates, ascorbates, and the like.

As used herein, the term “prodrug” refers to any covalently bonded substituent that results in the release of the active compound under physiological conditions following administration to a patient.

As used herein, the term “combination therapy” refers to a treatment methodology in which a patient is treated with, or given, another drug for the disease in conjunction with imexon. The combination therapy can be sequential therapy where the patient is treated first with one drug and then with the other(s), or the drugs can be given simultaneously.

As used herein, “cancer treatment regimen” refers to activities, the design and/or objective of which is the treatment of cancer, including inhibition or prevention of the growth of cancer, and reducing the growth of cancer relative to the absence of a therapy or treatment.

As used herein, the term “cancer” refers to all types of cancer, neoplasm, or malignant tumors found in mammals, including carcinomas, sarcomas, melanomas, plasma cell neoplasms, lymphomas, and leukemias.

As used herein, “carcinoma” refers to malignant tumors of epithelial origin.

As used herein, “sarcoma” refers to malignant neoplasms arising in tissue of mesodermal origin such as connective tissue, bone, cartilage, or striated muscle.

As used herein, “melanoma” refers to malignant tumors arising from the melanocytic system of the skin or other organs.

As used herein, “plasma cell neoplasm” refers to malignant diseases of plasma cells characterized by clonal proliferation and accumulation of immunoglobulin-producing cells that are terminally differentiated B cells.

As used herein, “leukemia” refers to malignant diseases of the blood-forming organs characterized by a distorted proliferation and development of leukocytes in the tissues of the body with or without a corresponding increase of leukocytes in the circulating blood.

As used herein, “lymphoma” refers to malignant tumors of lymphoid tissue.

As used herein, “constipation” refers to a gastrointestinal condition including an abnormally delayed, infrequent, irregular, or difficult evacuation of the bowels, and is intended to encompass occasional or intermittent symptoms, persistent symptoms, symptoms interfering with activities of daily living, life-threatening consequences (e.g., obstruction and toxic megacolon), and death. The term “imexon-induced constipation” refers to constipation that occurs in a patient as a result of imexon administration, including an increase in the severity of constipation as a result of imexon administration. The methods of treating imexon-induced constipation disclosed herein are not intended to include (provisos out) laxative administration to relieve existing constipation arising from causes other than administration of imexon, such as from treatment with other drugs, diet, lack of exercise, or unrelated gastrointestinal problems.

As used herein, the term “laxative” refers to oral and rectal preparations, the administration of which to a patient is intended to produce and/or encourage a bowel movement.

As used herein, the term “oral laxative” refers to a laxative administered to a patient by mouth, and includes bulk-forming laxatives, hyperosmotic laxatives, lubricant laxatives, stimulant laxatives, emollients, and combinations thereof.

As used herein, the term “rectal laxative” refers to a laxative, including an enema or suppository, administered to a patient via insertion into the rectum or anus, and includes carbon dioxide-releasing laxatives, hyperosmotic laxatives, lubricant laxatives, stimulant laxatives, emollients, and combinations thereof.

As used herein, the term “bulk-forming laxative” refers to a preparation that, upon administration to a patient, is principally not digested, but absorbs liquid in the intestines and swells to form a soft, bulky stool. Examples of bulk-forming laxatives include malt soup extract, malt soup extract and psyllium, methylcellulose, polycarbophil, psyllium, psyllium hydrophilic mucilloid, psyllium hydrophilic mucilloid and carboxymethylcellulose.

As used herein, the term “carbon dioxide-releasing laxative” refers to a preparation that, upon administration to a patient, generates carbon dioxide gas within the intestine. Examples of carbon dioxide-releasing laxatives include potassium bitartrate, and sodium bicarbonate.

As used herein, the term “hyperosmotic laxative” refers to a preparation that, upon administration to a patient, draws water into the bowel from surrounding tissues. Hyperosmotic laxatives include, for example, glycerin, as well as hyperosmotic saline laxatives (e.g., magnesium citrate, magnesium hydroxide, magnesium oxide, magnesium sulfate, sodium phosphate, and sodium biphosphate), hyperosmotic lactulose laxatives (e.g., lactulose), and hyperosmotic polymer laxatives (e.g., polyethylene glycol 3350).

As used herein, the term “lubricant laxative” refers to a preparation that, upon administration to a patient, coats the bowel and the stool mass with a film that maintains moisture in the stool. Lubricant laxatives include, for example, mineral oil.

As used herein, the term “stimulant laxative” refers to a preparation that, upon administration to a patient, acts on the intestinal wall to increase peristalsis of the intestine. Examples of stimulant laxatives include bisacodyl, casanthranol, cascara sagrada, cascara sagrada and bisacodyl, cascara sagrada and aloe, castor oil, dehydrocholic acid, senna, and sennosides.

As used herein, the term “emollient” refers to a preparation that, upon administration to a patient, softens the stool by aiding the mixing of liquids with the stool mass. Examples of emollients include docusates, and poloxamer 188.

As used herein, the term “about” is intended to indicate a range (e.g., ±10%) caused by experimental uncertainty, or by uncertainty associated with the measurement of a patient's body surface area, variation within the parameters of a label claim associated with a drug product, and variability in partitioning and/or dispensing a drug.

II. Cancer Treatment Regimens

It has been found that, unexpectedly, the administration to patients as part of a cancer treatment regimen, of a therapeutically effective amount of a pharmaceutical composition comprising imexon, induces constipation in a large percentage of patients. In one aspect, the present invention provides methods of treating constipation in a patient receiving imexon, for example in association with the patient's participation in a cancer treatment regimen. In another aspect, the present invention provides pharmaceutical kits comprising instructions to treat constipation as an event arising in association with the use of a pharmaceutical composition comprising imexon in a cancer treatment regimen.

Cancer treatment regimens with which the present invention is useful include those in which, alone or in combination therapy, a therapeutically effective amount of a pharmaceutical composition comprising imexon is administered to a patient in need thereof. Cancer treatment regimens in accordance with aspects of the present invention include those comprising a therapy to treat a cancer selected from carcinoma, sarcoma, melanoma, plasma cell neoplasm, lymphoma, or leukemia. More specifically, cancer treatment regimens in accordance with aspects of the present invention include those comprising a therapy to treat a cancer selected from multiple myeloma, malignant melanoma, colon cancer, bladder cancer, breast cancer, lung cancer, liver cancer, prostate cancer, ovarian cancer, endometrial cancer, thyroid cancer, renal cell cancer, or pancreatic cancer.

It will be appreciated that the foregoing represent examples and/or broad categories of cancers that may be a target of therapy within a patient's cancer treatment regimen, and are not intended to specifically limit the scope of embodiments of the present invention. Moreover, it will be appreciated that in some cancer treatment regimens in accordance with aspects of the present invention, the therapy may be designed and/or intended to treat multiple cancers in a single individual.

A. Pharmaceutical Compositions Useful in the Treatment of Cancer

Pharmaceutical compositions comprising imexon are useful in the treatment of cancer. Imexon has both direct cytotoxicity to tumor cells and immunostimulatory effects. In phase I human clinical trials conducted in Europe in 1985, imexon was well tolerated and produced objective responses or stabilized neoplastic diseases in cancer patients. (Sagaster et al.: J. Natl Cancer Inst 87:935, 1995). In some embodiments, the present invention provides a pharmaceutical composition including imexon (e.g., a therapeutically effective amount of imexon) and a laxative (e.g., in an amount effective to treat imexon-induced constipation).

More recently, preclinical investigations have shown imexon to be useful in combination therapy, and to exhibit a synergistic therapeutic cytotoxic effect when combined with antineoplastic nucleic acid binding agents such as cisplatin, dacarbazine, and melphalan, with antineoplastic antimetabolite base analogs such as cytarabine, 5-fluorouracil, and gemcitabine, and with docetaxel. A description of the synergistic therapeutic cytotoxic effect of combinations of cancer chemotherapeutic agents with imexon may be found in co-pending US patent application, Publication No. 2005/0176696 A1, titled, “SYNERGISTIC ANTI-CANCER COMPOSITIONS,” hereby incorporated by reference in its entirety. It will be appreciated that any one or more of these synergistic cancer chemotherapeutic agents may be employed as a component of a combination therapy with pharmaceutical compositions comprising imexon in accordance with aspects of the present invention.

In addition to the foregoing, other cancer chemotherapeutic agents that may be employed as a component of a combination therapy with a pharmaceutical composition comprising imexon include, but are not limited to, busulfan, cyclophosphamide, carmustine, mechlorethamine, thiotepa, chlorambucil, lomustine, ifosfamide, mitomycin C, carboplatin, oxaliplatin, temozolamide, semustine, porfiromycin, mercaptopurine, thioguanine, azathioprine, fludarabine, cladribine, pentostatin, fluorouracil, capecitabine, floxuridine, bleomycin, etoposide, vincristine, vinblastine, vinorelbine, topotecan, teniposide, and mitoxantrone.

Whether a particular cancer chemotherapeutic agent will be useful in combination therapy with imexon will be a function of the particular type of cancer being treated, and the patient's tolerance for the toxicity of the combination of chemotherapeutic agents, as well as other factors that will be familiar to the healthcare professional administering the cancer treatment regimen.

B. Dosage Forms

Pharmaceutical compositions that may be administered as a component of a cancer treatment regimen in accordance with aspects of the present invention can comprise any suitable dosage form, and can comprise a single compound or mixtures of two or more cancer chemotherapeutic agents. The pharmaceutical compositions can comprise, in combination with the active chemotherapeutic agent, solid or liquid pharmaceutically acceptable carriers chosen based on the type of administration being used, as well as other suitable components (e.g., binders, lubricants, buffering agents, stabilizing agents, and the like) known to be useful in the preparation of pharmaceutical dosage forms. Specific examples of pharmaceutically acceptable carriers and other suitable components that can be used to formulate dosage forms are well known to those skilled in the art, and are described in Remington's Pharmaceutical Sciences, Mack Publishing Company, a standard reference text in this field.

Pharmaceutical compositions comprising imexon, and optionally comprising chemotherapeutic agents useful as a component of a combination therapy with imexon and/or a laxative, can be administered in oral dosage forms such as tablets, capsules, pills, powders, granules, elixirs, tinctures, suspensions, syrups, or emulsions. In addition, the pharmaceutical compositions comprising components of a cancer treatment regimen in accordance with aspects of the present invention can also be administered in intravenous (bolus or infusion), intraperitoneal, subcutaneous, or intramuscular form, in each case using dosage forms well known to those of ordinary skill in the pharmaceutical arts.

It will be appreciated that the method of administration can be any suitable method that is effective in the treatment of the particular cancer or tumor type being treated in conjunction with the cancer treatment regimen. The treatment may be administered orally, rectally, topically, parenterally, intravenously, or via injection into the tumor or cancer. The method of administration of a therapeutically effective amount of the relevant cancer chemotherapeutic agent may vary depending on the type of disease being treated.

It is contemplated that parenteral treatment by intravenous, subcutaneous, or intramuscular application of a pharmaceutical composition comprising imexon, formulated with an appropriate carrier to facilitate application, will be the preferred method of administering imexon compositions in accordance with aspects of the present invention. Dosage forms useful for the administration of pharmaceutical compositions comprising imexon include for example, a parenteral composition suitable for administration by injection, the composition comprising 100 mg of imexon and 100 mg of mannitol, reconstituted with 20 ml of sterile water for injection.

Dosage forms useful for the administration of selected cancer chemotherapeutic agents that may be employed in combination therapy with pharmaceutical compositions comprising imexon include the following representative examples. Gemcitabine may be supplied as a parenteral composition suitable for administration by injection, the composition comprising 200 mg of gemcitabine HCl for reconstitution with 0.9% sterile sodium chloride to achieve a concentration of 38 mg/ml. A gemcitabine composition may be obtained from Eli Lilly and Company. Dacarbazine may be supplied as a parenteral composition suitable for administration by injection, the composition comprising 100 mg of dacarbazine, anhydrous citric acid, and mannitol for reconstitution with 9.9 ml of sterile water for injection to yield a solution of 10 mg/ml, which may be further diluted with 5% dextrose for injection or sodium chloride for injection. A dacarbazine composition may be obtained from Bayer Pharmaceuticals Corporation. Docetaxel may be supplied as a parenteral composition suitable for administration by injection, the composition comprising 80 mg of docetaxel in 2 ml polysorbate 80 for dilution with a suitable diluent to yield a solution having a concentration of docetaxel of about 10 mg/ml. A docetaxel composition may be obtained from Aventis Pharmaceuticals, Inc.

C. Dosage and Treatment Schedule

The dose of a chemotherapeutic agent that is administered to the patient will vary according to the specific drug being administered, the type of disease being treated, and the amount and kind of combination therapy, if any, being administered to the patient. One convention for describing the dose of a drug to be administered to a patient is in units of mass per units of body surface area (e.g., mg/m²).

In one embodiment, aspects of the present invention are useful in treating constipation arising in association with the administration of pharmaceutical compositions comprising imexon to a patient in a cancer treatment regimen to deliver a dose of imexon of from about 20 mg/m² to about 1300 mg/m² of body surface area. In another embodiment, aspects of the present invention are useful in treating constipation arising in conjunction with the administration of a pharmaceutical composition comprising imexon to a patient in a cancer treatment regimen to deliver a dose of imexon in an amount greater than or equivalent to about 185 mg/m² of body surface area. In yet another embodiment, aspects of the present invention are useful in treating constipation arising in conjunction with the administration of a pharmaceutical composition comprising imexon to a patient in a cancer treatment regimen to deliver a dose of imexon in an amount greater than or equivalent to about 245 mg/m² of body surface area.

In one embodiment in accordance with aspects of the present invention, the pharmaceutical composition comprising imexon, which is suitable for administration to a patient as part of a cancer treatment regimen, can include an appropriate amount of material withdrawn from an imexon composition comprising 100 mg of imexon and 100 mg of mannitol reconstituted in 20 ml of sterile water for injection, which is then aseptically introduced into a 250 ml or 500 ml bag of sterile sodium chloride for injection, and administered to the patient via infusion over a period of about 30 minutes (250 ml) or about 45 minutes (500 ml).

Treatment schedules for the administration of cancer chemotherapeutic agents to patients as part of a cancer treatment regimen conventionally comprise cycles of treatment wherein a specified dose of the chemotherapeutic agent(s) is/are administered to the patient at defined intervals over the period of a cycle, and then repeated in each subsequent cycle. The period of a cycle may be defined in any suitable manner, and may comprise, for example, a twenty-one day or three-week cycle, a twenty-eight day or four-week cycle, or the like. Within the period of a cycle of treatment, the specified dose of a chemotherapeutic agent can be administered to the patient at defined intervals, such as for example, for five consecutive days every other week (e.g., days 1-5 and 15-19 of a 28-day cycle), for five consecutive days every three weeks (e.g., days 1-5 of a 21-day cycle) once per week (e.g., days 1, 8, and 15 of a 21-day cycle), or the like, as determined with reference to each particular cancer chemotherapeutic agent being administered as part of the cancer treatment regimen.

The treatment schedule for the administration of a pharmaceutical composition comprising imexon can, in an embodiment in accordance with aspects of the present invention, comprise the daily administration of the specified dose, five days per week, every other week in a four-week cycle (i.e., days 1-5 and 15-19 of a 28 day cycle). It will be appreciated that other treatment schedules may be selected in accordance with the particular objectives of a cancer treatment regimen, and that the treatment schedules provided herein are not intended to be limiting. Furthermore, a cancer treatment regimen in accordance with aspects of the present invention may include one or more partial or complete cycles of administration of a chemotherapeutic agent. The number and completeness of such cycles of administration will be dependent upon the fulfillment of objectives of the cancer treatment regimen, the patient's health and tolerance for the administered agents, and other factors that will be familiar to one skilled in the art.

It will be appreciated that the total daily dose of a chemotherapeutic agent may be delivered in a single dose, or may be administered in a series of divided doses of two, three, four, or more times daily. Moreover, in the event that the cancer treatment regimen comprises a combination therapy, the two or more cancer chemotherapeutic agents can be administered to the patient sequentially or simultaneously, as appropriate under the particular circumstances of the cancer treatment regimen. Sequential therapy can be within a reasonable time after the administration of the first component of a combination therapy. Simultaneous treatment can comprise the treatment with two or more agents in the same dose or in separate doses.

While it will be appreciated that the dose and treatment schedule for administration of a specific cancer chemotherapeutic agent to be administered to a patient in a cancer treatment regimen may vary with the type of cancer being treated and the particular combination therapy being administered, the following are examples of a standard dose and schedule for representative chemotherapeutic agents useful in combination therapy with pharmaceutical compositions comprising imexon.

In one embodiment in accordance with aspects of the present invention, a pharmaceutical composition comprising imexon may be administered to a patient as one component of a combination therapy in the patient's cancer treatment regimen, which includes gemcitabine as a second component of the combination therapy. The pharmaceutical composition comprising imexon may, in an embodiment in accordance with aspects of the present invention, be administered at a dose of from about 100 mg/m² to about 1300 mg/m² of body surface area on a treatment schedule wherein the dose is administered five days per week, every other week in a four-week cycle. The combination therapy including a pharmaceutical composition comprising gemcitabine may, in an embodiment in accordance with aspects of the present invention, be administered at a dose of from about 600 mg/m² to about 1000 mg/m² of body surface area on a treatment schedule wherein the dose is administered once weekly for three consecutive weeks in a four-week cycle (i.e., days 1, 8, and 15 of a 28 day cycle).

In another embodiment in accordance with aspects of the present invention, a pharmaceutical composition comprising imexon may be administered to a patient as one component of a combination therapy in the patient's cancer treatment regimen, which includes dacarbazine as a second component of the combination therapy. The pharmaceutical composition comprising imexon may, in an embodiment in accordance with aspects of the present invention, be administered at a dose of from about 430 mg/m² to about 1300 mg/m² of body surface area on a treatment schedule wherein the dose is administered daily for five consecutive days in a three-week cycle (i.e., days 1-5 of a 21 day cycle). The combination therapy including a pharmaceutical composition comprising dacarbazine may, in an embodiment in accordance with aspects of the present invention, be administered at a dose of from about 190 mg/m² to about 250 mg/m² of body surface area on a treatment schedule wherein the dose is administered daily for five consecutive days in a three-week cycle. In one embodiment in accordance with aspects of the present invention the two chemotherapeutic agents can be administered sequentially.

In yet another embodiment in accordance with aspects of the present invention, a pharmaceutical composition comprising imexon may be administered to a patient as one component of a combination therapy in the patient's cancer treatment regimen, which includes docetaxel as a second component of the combination therapy. The pharmaceutical composition comprising imexon may, in an embodiment in accordance with aspects of the present invention, be administered at a dose of from about 250 mg/m² to about 1300 mg/m² of body surface area on a treatment schedule wherein the dose is administered daily for five consecutive days in a three-week cycle. The combination therapy including a pharmaceutical composition comprising docetaxel may, in an embodiment in accordance with aspects of the present invention, be administered at a dose of from about 55 mg/m² to about 100 mg/m² of body surface area on a treatment schedule wherein the dose is administered once in a three-week cycle (i.e., day 1 of a 21 day cycle).

D. Pharmaceutical Kits

The present invention includes pharmaceutical kits useful for the treatment of cancer. In some embodiments, the pharmaceutical kit includes a first pharmaceutical composition comprising imexon (e.g., a therapeutically effective amount to imexon) and a second pharmaceutical composition comprising a laxative (e.g., in an amount effective to treat imexon-induced constipation). Other embodiments of pharmaceutical kits in accordance with the present invention comprise one or more containers containing a pharmaceutical composition comprising a therapeutically effective amount of imexon, instructions to use the pharmaceutical composition in a cancer treatment regimen, and instructions to treat constipation arising in association with the use of the pharmaceutical composition in the cancer treatment regimen. In one embodiment, the pharmaceutical kit may include printed material identifying constipation as a gastrointestinal adverse reaction associated with use of imexon, and a recommendation to prescribe a prophylactic anticonstipation regimen in conjunction with administration of imexon to patients. Pharmaceutical kits in accordance with the present invention can further include, if desired, one or more of various conventional pharmaceutical kit components, such as, for example, containers with one or more pharmaceutically acceptable carriers, additional containers, and the like, as will be readily apparent to those skilled in the art.

The instructions for the use of the pharmaceutical composition in a cancer treatment regimen, and/or the instructions to treat constipation can, in various embodiments, comprise printed instructions in the form of an insert, a label, or other suitable mechanism of communicating information. The instructions can, in an embodiment, indicate quantities of the pharmaceutical composition(s), components of an anticonstipation regimen, or other components to be administered, guidelines for administration, and/or guidelines for mixing various components. It will be appreciated that additional unspecified materials, components, and/or instructions are not excluded so long as they do not prevent the benefits of the invention from being realized.

Pharmaceutical kits in accordance with embodiments of the present invention can include pharmaceutical compositions and instructions for their use in a cancer treatment regimen to treat a cancer selected from carcinoma, sarcoma, melanoma, plasma cell neoplasm, lymphoma, or leukemia. More specifically, pharmaceutical kits in accordance with embodiments of the present invention can include compositions and instructions for their use in a cancer treatment regimen to treat a cancer selected from multiple myeloma, malignant melanoma, colon cancer, bladder cancer, breast cancer, lung cancer, liver cancer, prostate cancer, ovarian cancer, endometrial cancer, thyroid cancer, renal cell cancer, or pancreatic cancer.

It will be appreciated that the pharmaceutical kits in accordance with embodiments of the present invention can include any one or more of the pharmaceutical compositions described herein, including those useful in combination therapy, in any suitable dosage form useful to provide a therapy in a cancer treatment regimen in accordance with aspects of the present invention. Moreover, embodiments of pharmaceutical kits may include laxatives or other components of an anticonstipation regimen and/or instructions for their use or other instructions useful in treating constipation arising in association with the use of pharmaceutical compositions comprising imexon in a cancer treatment regimen. A more detailed description of anticonstipation regimens and components thereof in accordance with the present invention is provided hereinafter.

III. Clinical Management of Patients

As part of a cancer treatment regimen, patients are periodically assessed to evaluate the status of their disease and their tolerance for the cancer chemotherapeutic agents being administered as part of their cancer treatment regimen. Disease assessments are commonly performed at intervals corresponding to the patient's completion of a specified number of treatment cycles, and often include evaluations via direct physical palpation of the tumor mass, or by means of an objective scan such as with x-ray radiographs, computerized tomography (CAT scans), or magnetic resonance imaging (MRI). In addition, the tumor may secrete a marker substance that may be indicative of tumor load for specific types of cancers, and a measurement of the levels of these substances may be useful in assessing a patient's disease progression or response to their cancer treatment regimen.

An assessment of a patient's tolerance for a cancer chemotherapeutic agent, or combinations thereof, is typically performed with each course of chemotherapy (i.e., each time a dose is administered to the patient), and is intended to identify toxicities (also commonly referred to as adverse events) associated with the administration of the pharmaceutical composition comprising the chemotherapeutic agent, and with the patient's overall participation in the cancer treatment regimen. The identification of toxicities associated with the administration of a chemotherapeutic agent is important to ensure that appropriate treatments or remedial measures can be administered or otherwise undertaken to prevent or alleviate the toxicity and provide the greatest safety for the patient during their participation in a cancer treatment regimen.

Chemotherapeutic toxicities or adverse events are defined by organizations such as the National Cancer Institute, which provides a periodically revised listing of descriptive terminology categorizing adverse events based on anatomy and/or pathophysiology (e.g., Blood/Bone Marrow, Gastrointestinal, Renal/Genitourinary, and the like). See, e.g., Common Terminology Criteria for Adverse Events, published by the U.S. Department of Health and Human Services, NIH, NCI. In addition, the adverse events are categorized with reference to a grade that is indicative of the severity of the adverse event, and wherein grade 1 refers to a mild event, grade 2 refers to a moderate event, grade 3 refers to a severe event, grade 4 refers to a life-threatening or disabling event, and grade 5 refers to death related to the adverse event. This compilation of common criteria provides a means of accurately identifying toxicities associated with the use of a cancer chemotherapeutic agent so that protocols for the treatment and/or prevention of such events can be provided to enhance the safety of a chemotherapeutic agent's use in a cancer treatment regimen.

An adverse event is broadly defined as any untoward medical occurrence in a patient regardless of causal relationship to treatment, and may include any unfavorable sign, symptom, or disease temporally associated with the use of a chemotherapeutic agent. Although all adverse events arising in conjunction with the treatment of a patient with a chemotherapeutic agent are reported as a component of clinical protocols that define parameters for the investigation of a drug's safety and efficacy, the adverse events are typically classified by the treatment provider substantially as “not related to the treatment,” “unlikely to be related to the treatment,” “possibly related to the treatment,” “probably related to the treatment,” or “definitely related to the treatment.” The classification of an adverse event in regard to its relationship to the treatment of a patient with a chemotherapeutic agent is a function of the patient's medical history, the use of any concomitant medications, and other factors that the healthcare provider evaluates in order to make a judgment about the likely relationship between the observed toxicity and the patient's treatment with a chemotherapeutic agent in the course of a cancer treatment regimen. While determining the specific causal relationship between the administration of a chemotherapeutic agent and observed toxicity is difficult, there is often a distinction made between those adverse events characterized as “not,” or “unlikely to be related to the treatment,” and those adverse events characterized as “possibly,” “probably,” or “definitely related to the treatment,” giving greater significance to those having the later characterization.

A. Assessment and Management of Constipation

As described hereinbefore, it has been found, unexpectedly, that the administration of a pharmaceutical composition comprising imexon induces constipation in a large percentage of patients. Of forty nine (49) patients taking part in a clinical study investigating the use of imexon to treat cancer, thirty one (31) patients (63.3%) were reported to have had the gastrointestinal toxicity of constipation in association with their treatment with imexon. Constipation adverse events comprised 21.8% of all gastrointestinal adverse events observed in the forty nine (49) patients, and accounted for a greater percentage of total gastrointestinal adverse events than did any other single gastrointestinal adverse event, with the exception of nausea, which accounted for 23.0% of all gastrointestinal adverse events. The next most commonly observed gastrointestinal adverse events were anorexia (13.4% of total gastrointestinal adverse events), vomiting (11.9% of total gastrointestinal adverse events), and diarrhea (10.5% of total gastrointestinal adverse events). All other observed gastrointestinal adverse events individually accounted for less than 5% of total gastrointestinal adverse events.

Of the forty nine (49) patients treated in the clinical study, thirty nine (39) received imexon at a dose equal to or greater than 140 mg/m², thirty three (33) received imexon at a dose equal to or greater than 185 mg/m², and thirty (30) received imexon at a dose equal to or greater than 245 mg/m². Of the thirty nine (39) patients receiving imexon at a dose equal to or greater than 140 mg/m², twenty five (25) patients (64.1%) were reported to have had the gastrointestinal toxicity of constipation in association with their treatment with imexon. Of the thirty three (33) patients receiving imexon at a dose equal to or greater than 185 mg/m², twenty two (22) patients (66.7%) were reported to have had the gastrointestinal toxicity of constipation in association with their treatment with imexon. Of the thirty (30) patients receiving imexon at a dose equal to or greater than 245 mg/m², twenty (20) patients (66.7%) were reported to have had the gastrointestinal toxicity of constipation in association with their treatment with imexon.

Following the unexpected identification of constipation toxicities in patients being treated with imexon in a cancer treatment regimen, a GLP (good laboratory practices) study was undertaken to evaluate the effects of imexon on the gastrointestinal transit of a charcoal meal in rats. The results of the study indicated that the intravenous administration of imexon caused a statistically significant inhibition of gastrointestinal transit and a reduction in gastric emptying in rats at a dose of 100 mg/kg (roughly equivalent to 529 mg/m² in an adult human). These results confirmed the unexpected identification of constipation in the patients treated with imexon.

As illustrated in Table 1, a total of seventy three (73) adverse events characterized as constipation were reported in the forty nine (49) patients receiving treatment with imexon at doses ranging from 20 mg/m² to 1000 mg/m² of body surface area on a schedule comprising daily administration of the specified dose for five consecutive days per week, every other week, in a four-week cycle.

TABLE 1
Constipation Adverse Events

| No. | Imexon | | Grade | | | | | |
|---|---|---|---|---|---|---|---|---|
| Patients | Dose (mg/m²) | Relationship to Treatment | 1 | 2 | 3 | 4 | 5 | Total |
| 1 | 20 | Definite | | | | | | 0 |
| | | Probable | | | | | | 0 |
| | | Possible | | | | | | 0 |
| | | Unlikely | | | | | | 0 |
| | | Not Related | | 1 | 1 | | | 2 |
| 1 | 40 | Definite | | | | | | 0 |
| | | Probable | | | | | | 0 |
| | | Possible | | | | | | 0 |
| | | Unlikely | | | | | | 0 |
| | | Not Related | | | | | | 0 |
| 5 | 80 | Definite | | | | | | 0 |
| | | Probable | | | | | | 0 |
| | | Possible | | | | | | 0 |
| | | Unlikely | | | | | | 0 |
| | | Not Related | | 3 | 1 | | | 4 |
| 3 | 105 | Definite | | | | | | 0 |
| | | Probable | | | | | | 0 |
| | | Possible | | | | | | 0 |
| | | Unlikely | | | | | | 0 |
| | | Not Related | | 5 | 3 | | | 8 |
| 6 | 140 | Definite | | | | | | 0 |
| | | Probable | | 1 | | | | 1 |
| | | Possible | | | | | | 0 |
| | | Unlikely | | 2 | 1 | | | 3 |
| | | Not Related | | 1 | | | | 1 |
| 3 | 185 | Definite | | | | | | 0 |
| | | Probable | | 1 | | | | 1 |
| | | Possible | 1 | | | | | 1 |
| | | Unlikely | | | | | | 0 |
| | | Not Related | | | 1 | | | 1 |
| 3 | 245 | Definite | | | | | | 0 |
| | | Probable | | 2 | 1 | | | 3 |
| | | Possible | | 4 | 1 | | | 5 |
| | | Unlikely | | | | | | 0 |
| | | Not Related | 1 | | | | | 1 |
| 4 | 325 | Definite | | | | | | 0 |
| | | Probable | 1 | 2 | | | | 3 |
| | | Possible | | 2 | | | | 2 |
| | | Unlikely | | | | | | 0 |
| | | Not Related | | | | | | 0 |
| 3 | 430 | Definite | | | | | | 0 |
| | | Probable | 2 | 5 | 1 | | | 8 |
| | | Possible | | 1 | | | | 1 |
| | | Unlikely | 1 | | | | | 1 |
| | | Not Related | | | | | | 0 |
| 3 | 570 | Definite | 1 | 4 | 2 | | | 7 |
| | | Probable | 1 | | | | | 1 |
| | | Possible | | | | | | 0 |
| | | Unlikely | | | | | | 0 |
| | | Not Related | | | | | | 0 |
| 6 | 750 | Definite | 1 | 4 | 3 | | | 8 |
| | | Probable | | 2 | | | | 2 |
| | | Possible | 2 | | | | | 2 |
| | | Unlikely | | | | | | 0 |
| | | Not Related | | | | | | 0 |
| 9 | 875 | Definite | 2 | 1 | 1 | | | 4 |
| | | Probable | 1 | | | | | 1 |
| | | Possible | | 1 | | | | 1 |
| | | Unlikely | | | | | | 0 |
| | | Not Related | | | | | | 0 |
| 2 | 1000 | Definite | | | | | | 0 |
| | | Probable | | 1 | | | | 1 |
| | | Possible | | | | | | 0 |
| | | Unlikely | | | | | | 0 |
| | | Not Related | | | | | | 0 |
| — | — | (Total) Definite | 4 | 9 | 6 | 0 | 0 | (Total) 19 |
| | | (Total) Probable | 5 | 14 | 2 | 0 | 0 | (Total) 21 |
| | | (Total) Possible | 3 | 8 | 1 | 0 | 0 | (Total) 12 |
| | | (Total) Unlikely | 1 | 2 | 1 | 0 | 0 | (Total) 4 |
| | | (Total) Not Related | 1 | 10 | 6 | 0 | 0 | (Total) 17 |
| (Total) | — | — | (Total) 14 | (Total) 43 | (Total) 16 | (Total) 0 | (Total) 0 | (Total) 73 |
| 49 | | | | | | | | |

Of these seventy three (73) constipation adverse events, fifty two (52) of the adverse events (71.2%) were characterized as “possibly,” “probably,” or “definitely related to the treatment” of the patient with imexon. Moreover, of the adverse events characterized as constipation that were reported in patients receiving imexon at a dose equal to or greater than 185 mg/m² (54 constipation adverse events in total), 94.4% were characterized as “possibly,” “probably,” or “definitely related to the treatment” of the patient with imexon. Furthermore, of the adverse events characterized as constipation that were reported in patients receiving imexon at a dose equal to or greater than 245 mg/m² (51 constipation adverse events in total) 96.1% were characterized as “possibly,” “probably,” or “definitely related to the treatment” of the patient with imexon. Of particular significance, 72.2% of adverse events characterized as constipation that were reported in patients receiving imexon at a dose equal to or greater than 185 mg/m² were characterized as “probably,” or “definitely related to the treatment” of the patient with imexon, and 74.5% of adverse events characterized as constipation that were reported in patients receiving imexon at a dose equal to or greater than 245 mg/m² were characterized as “probably,” or “definitely related to the treatment” of the patient with imexon.

Constipation arising in association with a patient's participation in a cancer treatment regimen including the administration of a pharmaceutical composition comprising imexon may be treated in accordance with embodiments of the present invention in a manner consistent with the severity of the observed symptoms. For example, for grade 1 constipation adverse events comprising occasional or intermittent symptoms, the occasional use of laxatives and/or a dietary modification may be prescribed and/or administered, in an embodiment. For persistent symptoms comprising a grade 2 constipation adverse event, regular use of laxatives may be prescribed and/or administered, in an embodiment. For grade 3 constipation adverse events comprising symptoms that interfere with activities of daily living, and including obstipation, regular use of laxatives and/or manual evacuation may be prescribed and/or administered, in an embodiment. In those patients in which a grade 4 constipation adverse event is observed, and wherein the symptoms comprise life-threatening consequences, the regular use of laxatives and particularly rectal laxatives including enemas may be prescribed and/or administered, in an embodiment.

As used hereinbefore and throughout the specification and claims, the term “prescribe” in any of its forms (e.g., prescribing, prescribed, or the like), with reference to an anticonstipation regimen, is intended to encompass an act of writing or giving a medical prescription for one or more components of an anticonstipation regimen, an act of administering one or more components of an anticonstipation regimen to a patient, an act of instructing a patient in the use of or to use one or more components of an anticonstipation regimen, an act of recommending the use of one or more components of an anticonstipation regimen, or any combination of the foregoing. This definition is not intended to detract from the scope of other language used in reference to anticonstipation regimens of the present invention, but rather to supplement and clarify the reader's understanding of characteristics of embodiments of the present invention.

B. Anticonstipation Regimens

Anticonstipation regimens in accordance with embodiments of the present invention may be designed to prevent the occurrence of constipation toxicities in patients being treated with imexon as part of a cancer treatment regimen. In other embodiments in accordance with the present invention, anticonstipation regimens may be designed to diminish the severity of symptoms comprising constipation toxicities in patients being treated with imexon as part of a cancer treatment regimen, as compared to symptoms in patients in which no such anticonstipation regimen is administered. Such prophylactic anticonstipation regimens may be administered or prescribed prior to, or at any time during, the patient's participation in a cancer treatment regimen, which includes the administration of a pharmaceutical composition comprising imexon. In one embodiment in accordance with the present invention, a prophylactic anticonstipation regimen can be administered and/or prescribed in conjunction with the administration of the first cycle of the therapeutically effective amount of the pharmaceutical composition including imexon. In still other embodiments in accordance with the present invention, anticonstipation regimens may be designed to treat constipation occurring in conjunction with the cancer treatment regimen, wherein the symptoms corresponding to the constipation are evident prior to the administration and/or prescription of the anticonstipation regimen.

As used throughout the specification and claims, the term “treat,” in any of its forms (e.g., treating, treated, or the like), with reference to constipation, is intended to encompass the prevention of the occurrence of constipation events/symptoms in patients, the diminishment of the severity of constipation events/symptoms, the resolution of constipation events/symptoms, or any combination of the foregoing.

Anticonstipation regimens in accordance with embodiments of the present invention can include oral and/or rectal laxatives alone or in combination with other components such as dietary modifications or other procedures (e.g., manual evacuation) as desired and/or necessary to treat a patient's constipation arising in association with the patient's participation in a cancer treatment regimen that includes the administration of a pharmaceutical composition comprising imexon. Oral and/or rectal laxatives useful as components of anticonstipation regimens in accordance with embodiments of the present invention include bulk-forming laxatives, carbon dioxide-releasing laxatives, hyperosmotic laxatives, lubricant laxatives, stimulant laxatives, emollients, and/or combinations thereof.

Commercially available products comprising one or more of the foregoing components include, for example, Metamucil® fiber laxative (comprising a psyllium husk, Procter & Gamble), Colace® capsules or liquid solution (comprising docusate sodium, Purdue Products L.P.), Senekot® tablets (comprising sennosides, Purdue Products L.P.), Perdiem® (comprising sennosides, Novartis Consumer Healthcare, Inc.), Kristalose™ (comprising lactulose, Bertek Pharmaceuticals, Inc.), Fleet® phospho-soda® (comprising sodium phosphate, C.B. Fleet, Inc.), Fleet® bisacodyl laxatives (comprising bisacodyl, C.B. Fleet, Inc.), Fleet® glycerin laxatives (comprising glycerin, C.B. Fleet, Inc.), Miralax™ (comprising polyethylene glycol 3350, Braintree Laboratories, Inc.), Senekot-S® tablets (comprising sennosides and docusate sodium, Purdue Products, L.P.), Fleet® mineral oil enema (comprising mineral oil, C.B. Fleet, Inc.), and Peri-Colace® tablets (comprising docusate sodium and senna, Purdue Products L.P.).

It will be appreciated that the particular type of laxative, dose, and schedule of administration may depend on the particular symptoms experienced by a patient, and the severity thereof. A determination by a healthcare provider to modify a particular anticonstipation regimen to address unusual or unique symptoms or responses in individual patients is contemplated by the present disclosure, and is intended to be encompassed by embodiments of the present invention. Anticonstipation regimens in accordance with embodiments of the present invention may be administered to the patient by a healthcare provider, or may be prescribed to the patient with the intention that the patient himself or herself administer the anticonstipation regimen or components thereof. Where the anticonstipation regimen is prescribed to the patient, the prescription may comprise instructions to obtain and/or administer components of the anticonstipation regimen with or without regard to the identification of symptoms by the patient, in an embodiment.

The instructions to administer components of the anticonstipation regimen may, in one embodiment, comprise instructions on the use or application of one or more components of the anticonstipation regimen. In another embodiment, the instructions to administer components of the anticonstipation regimen may comprise instructions that one or more components of the anticonstipation regimen is recommended and that the patient should administer the identified component(s) in accordance with instructions provided with the product.

Anticonstipation regimens in accordance with embodiments of the present invention may include one or more components as described previously, along with specified dosing and administration schedule instructions. In one embodiment, the anticonstipation regimen may comprise a series of treatment actions, each including one or more laxatives or other components along with dosing and administration schedule instructions. It will be appreciated that, in various embodiments, the subsequent actions in a series of treatment actions included in an anticonstipation regimen may comprise independent treatments (i.e., the treatment corresponding to the preceding action will be discontinued), or cumulative treatments, whereby the components of the subsequent treatment action are intended to be added to the continuing treatment corresponding to the preceding treatment action. The initiation of subsequent actions in a series of treatment actions may, in one embodiment, be dependent upon the response of the patient to a preceding action (e.g., if the constipation symptoms are successfully resolved following a treatment, any subsequent treatment actions may be disregarded).

In embodiments of the present invention, particularly those wherein subsequent treatment actions are dependent upon the response of the patient to a preceding action, the anticonstipation regimen may further include a particular objective goal (e.g., one bowel movement per day), that may be used to indicate the success of the particular treatment action, and/or to trigger the occurrence of subsequent treatment actions when the objective goal is not satisfied. In other embodiments, each of a series of treatment actions can be completed without reference to the presence or absence of constipation symptoms in accordance with instructions in the anticonstipation regimen. For example, this treatment scenario may be applicable in those instances in which the anticonstipation regimen is intended to prevent or curtail the constipation symptoms arising in association with the administration of a pharmaceutical composition comprising imexon, rather than to treat the occurrence of symptoms that have already been identified.

By way of illustrating the foregoing, one representative embodiment of an anticonstipation regimen in accordance with the present invention may comprise a series of four treatment actions. The first treatment action may, in an embodiment, include the prescription or administration of an emollient (e.g., a docusate sodium composition such as Colace® capsules) at a dose of about 200 mg per day in combination with an emollient/stimulant laxative (e.g., a combination sennosides and docusate sodium composition such as Senekot-S® tablets) at a dose of about 17.2 mg sennosides and 100 mg docusate sodium, twice per day, with the goal of one bowel movement per day. In one embodiment, the first treatment action may be initiated two days prior to administration of a course of imexon in a cycle of treatment (e.g., initiation of the treatment action on day −2 or day 13 where the cycle includes administration of imexon on days 1-5 and 15-19 of a 28-day cycle) within the patient's cancer treatment regimen. In one embodiment, the second treatment action may include the prescription or administration of a hyperosmotic polymer laxative (e.g., a polyethylene glycol 3350 composition such as Miralax™) at a dose of about 17 g, once per day, in addition to the first treatment action, with the goal of one bowel movement within a two-day period. The third treatment action may include, in an embodiment, the prescription or administration of the same component identified in the second treatment action, but on a schedule of twice per day, replacing (rather than adding to) the second treatment action. In one embodiment, the goal of the third treatment action may be to induce a bowel movement within the period ending with the third day of treatment with imexon (e.g., day 3 or day 17 of a 28-day cycle in which imexon is administered on days 1-5 and 15-19). The fourth treatment action may, in an embodiment, comprise the prescription or administration of a hyperosmotic saline laxative (e.g., a sodium phosphate composition such as Fleet® phospho-soda®) at a dose of about 20 g of sodium phosphate (e.g., 30 ml of Fleet® phospho-soda® contains about 14.4 g of monobasic sodium phosphate monohydrate, and about 5.4 g of dibasic sodium phosphate heptahydrate) every four hours, up to a total of four doses per day, until the constipation has resolved.

While the invention is described here in the context of a limited number of embodiments, and with reference to specific details and examples, the invention may be embodied in many forms without departing from the spirit of the essential characteristics of the invention. For instance, it will be appreciated that anticonstipation regimens in accordance with the present invention can vary by the number and type of components included, the dose and schedule of administration, and the inclusion, as well as number, of multiple treatment actions. The exemplary and described embodiments, including what is described in the abstract of the disclosure, are therefore to be considered in all respects as illustrative and not restrictive. The scope of the invention is indicated by the appended claims rather than by the foregoing description, and all changes that come within the meaning and range of equivalency of the claims are intended to be embraced therein.

What is claimed is:

1. A method of treating imexon-induced constipation in a patient in association with the patient's participation in a cancer treatment regimen, wherein the cancer treatment regimen includes administration of a therapeutically effective amount of a pharmaceutical composition including imexon, the method comprising:
prescribing an anticonstipation regimen to treat imexon-induced constipation occurring in conjunction with the cancer treatment regimen, the anticonstipation regimen comprising a laxative selected from a group consisting of bulk-forming laxatives, carbon dioxide-releasing laxatives, hyperosmotic laxatives, lubricant laxatives, stimulant laxatives, emollients, and combinations thereof.

2. A method of treating imexon-induced constipation in a patient being treated with imexon, the method comprising administering a laxative sufficient to treat the imexon-induced constipation.

3. The method of claim 2, wherein the laxative is selected from a group consisting of bulk-forming laxatives, carbon dioxide-releasing laxatives, hyperosmotic laxatives, lubricant laxatives, stimulant laxatives, and emollients.

4. The method of claim 1, wherein the pharmaceutical composition is administered to treat a cancer selected from a group consisting of carcinoma, sarcoma, melanoma, plasma cell neoplasm, lymphoma, and leukemia.

5. The method of claim 4, wherein the pharmaceutical composition is administered to treat a cancer selected from a group consisting of multiple myeloma, malignant melanoma, colon cancer, bladder cancer, breast cancer, lung cancer, liver cancer, prostate cancer, ovarian cancer, endometrial cancer, thyroid cancer, renal cell cancer, pancreatic cancer, and a non-Hodgkin's lymphoma.

6. The method of claim 1, wherein the administration of the pharmaceutical composition comprises a component of a combination therapy in the patient's cancer treatment regimen.

7. The method of claim 6, wherein the combination therapy includes administration of a therapeutically effective amount of a pharmaceutical composition comprising a chemotherapeutic agent selected from a group consisting of gemcitabine, dacarbazine, and docetaxel.

8. The method of one of claims 1, 2, or 3, wherein the laxative comprises an emollient in combination with a stimulant laxative.

9. The method of claim 8, wherein the laxative further comprises a hyperosmotic polymer laxative.

10. The method of claim 9, wherein the laxative further comprises a hyperosmotic saline laxative.

11. The method of claim 1, wherein the anticonstipation regimen comprises two or more treatment actions to treat constipation occurring in conjunction with the cancer treatment regimen.

12. The method of claim 11, wherein the two or more treatment actions include at least one cumulative treatment action.

13. The method of claim 1, wherein the pharmaceutical composition comprises a composition suitable for administration by injection.

14. The method of claim 1, wherein the pharmaceutical composition comprises a solid dosage form including imexon and a pharmaceutically acceptable carrier.

15. The method of claim 1, wherein the pharmaceutical composition comprises a liquid dosage form including imexon and a pharmaceutically acceptable carrier.

16. The method of one of claims 1 or 2, wherein the imexon is administered in an amount greater than or equivalent to about 185 mg/m² of body surface area.

17. The method of one of claims 1 or 2, wherein the imexon is administered in an amount greater than or equivalent to about 245 mg/m² of body surface area.

18. The method of claim 1, wherein the cancer treatment regimen comprises a dosing schedule providing for the administration of the pharmaceutical composition once weekly for three consecutive weeks in a four-week cycle.

19. The method of claim 1, wherein the cancer treatment regimen comprises a dosing schedule providing for the administration of the pharmaceutical composition for five consecutive days in a three-week cycle.

20. The method of claim 1, wherein the anticonstipation regimen comprises a prophylactic anticonstipation regimen prescribed in conjunction with the administration of a first cycle of the therapeutically effective amount of the pharmaceutical composition including imexon.

21. The method of claim 20, wherein the prophylactic anticonstipation regimen is initiated prior to administration of the first cycle of the therapeutically effective amount of the pharmaceutical composition including imexon.

22. A method of treating imexon-induced constipation in a patient being treated with imexon, the method comprising administering a laxative sufficient to treat the imexon-induced constipation, and wherein the treatment with imexon comprises administration of from about 800 mg/m² of body surface area per day to about 1200 mg/m² of body surface area per day for five consecutive days in a three-week cycle.

23. The method of claim 22, wherein the imexon is administered to treat a cancer selected from a group consisting of malignant melanoma, breast cancer, prostate cancer, and non-small cell lung cancer.

24. The method of claim 22, wherein the imexon is administered as a component of a combination therapy, the combination therapy including a second component selected from a group consisting of dacarbazine and docetaxel.

25. A method of treating imexon-induced constipation in a patient being treated with imexon, the method comprising administering a laxative sufficient to treat the imexon-induced constipation, and wherein treatment with imexon comprises administration of from about 350 mg/m² of body surface area per day to about 750 mg/m² of body surface area per day once weekly for three consecutive weeks in a four-week cycle.

26. The method of claim 25, wherein the imexon is administered to treat pancreatic adenocarcinoma.

27. The method of claim 25, wherein the imexon is administered as a component of a combination therapy, the combination therapy including a second component comprising gemcitabine.

28. A pharmaceutical composition comprising a therapeutically effective amount of imexon and an amount of a laxative effective in treating imexon-induced constipation.

29. The pharmaceutical composition of claim 28, wherein the laxative is selected from a group consisting of bulk-forming laxatives, carbon dioxide-releasing laxatives, hyperosmotic laxatives, lubricant laxatives, stimulant laxatives, and emollients.

30. The pharmaceutical composition of claim 28, wherein the pharmaceutical composition is an oral dosage form.
